(19) United States
(12) Patent Application Publication
(10) Pub. No.: US 2017/0086956 A1
(43) Pub. Date: Mar. 30, 2017

(54) Foldable Pessary for Treatment of Stress Urinary Incontinence

(71) Applicant: Wisconsin Alumni Research Foundation, Madison, WI (US)

(72) Inventors: Gloria E. Sarto, Madison, WI (US); Heidi Brown, Monona, WI (US); Mufaddal Lakdawala, Madison, WI (US); Joshua Plantz, Niantic, CT (US); Anneka Littler, Makinen, MN (US); Jack McGinnity, Madison, WI (US); Thomas Feustel, Fond du Lac, WI (US)

(21) Appl. No.: 14/870,471

(22) Filed: Sep. 30, 2015

(51) Int. Cl. A61F 2/00; A61F 6/08

(52) U.S. Cl. CPC A61F 2/0009; A61F 6/08; A61F 2230/0017; A61F 2250/0018; A61F 2250/0019; A61F 2250/0029

(57) ABSTRACT

The present invention provides a vaginal pessary that offers mid-urethral support and is supported laterally near the vaginal opening, rather than between the apex of the vagina and the back of the pubic bone as is found in current pessaries. The pessary is inserted closer to the opening of the vagina so that it will support the mid-urethra, rather than the bladder neck, in accordance with the new understanding of the importance of mid-urethral support. The pessary is smaller and applies minimal force along the inner walls of the vagina.

### STATEMENT REGARDING FEDERALLY SPONSORED RESEARCH OR DEVELOPMENT

--

### CROSS-REFERENCE TO RELATED APPLICATIONS

--

### BACKGROUND OF THE INVENTION

The present invention relates to vaginal pessaries for treating stress urinary incontinence.

Accidental urinary leakage affects more than half of independent women aged 65 and older in the United States. Stress urinary incontinence is the most prevalent type of urinary incontinence, present in over 80% of urinary incontinence cases. Stress urinary incontinence is defined by leakage associated with activities that increase intra-abdominal pressure, such as when coughing, sneezing, and during physical activity. Pelvic floor muscle strengthening, vaginal pessaries, and mid-urethral sling surgery are common treatment options for those suffering from stress urinary incontinence.

Pelvic floor muscle strengthening exercises are done by flexing the muscles used to stop urination. When exercises are maintained, pelvic floor muscle strengthening exercises are shown to be effective in treating stress urinary incontinence in just over half of women in the short term. However, many women discontinue their exercises over time.

Pessaries are vaginally inserted devices that are used to treat pelvic organ prolapsed as well as urinary incontinence. Current pessaries for treating urinary incontinence are commonly ring-shaped devices placed with one end against the cervix of the vagina and the more distal end against the top vaginal wall. The more distal end is intended to support the area beneath the bladder neck of the urethra. Current vaginal pessaries operate on the belief that loss of support of the bladder neck contributed to the cause of stress urinary incontinence. Current pessaries are shown to improve the symptoms of about half of women.

Through a better understanding of the underlying pathophysiology of stress urinary incontinence, it has been found that support of the urethra, not just the bladder neck, may be important for treating stress urinary incontinence. Mid-urethral sling surgery draws on this idea by using a synthetic mesh to provide “sling” support underneath the mid-urethra where the weakened pelvic floor muscles previously provided support. Mid-urethral sling surgery has proven to be the most effective treatment so far for treating stress urinary incontinence, resulting in improvement in over 90% of women who undergo the surgery.

Despite the success of mid-urethral sling surgery, the surgery may not be an available option to all women and others may not wish to undergo the invasive surgery.

### SUMMARY OF THE INVENTION

The present inventors have recognized that it is possible to produce a vaginal pessary that offers appropriate mid-urethral support and is supported laterally near the vaginal opening, rather than between the apex of the vagina and the back of the pubic bone as is found in current pessaries. The new vaginal pessary is inserted closer to the opening of the vagina so that it will support the mid-urethra, rather than the bladder neck, in accordance with the new understanding of the importance of mid-urethral support.

In one embodiment, the present invention may be a method of treating stress urinary incontinence having the steps of providing a pessary dimensioned to extend across opposing walls of a vagina and having a first end having a first support knob opposite a second end having a second support knob and a bridge extending along a first axis and arching along a second axis substantially perpendicular to the first axis between the first and second ends to provide a substantially incompressible structure configured to bend allowing pinching of the first and second ends together, pinching the first and second ends together to allow insertion into the vagina; inserting the pessary approximately 1-2 cm within the opening of the vagina; and releasing the first and second ends of the pessary while the pessary is in the vagina to allow the first and second support knobs to separate to contact opposing walls of the vagina and the bridge to arch toward a posterior sidewall of the vagina to allow an underside of the bridge to support the mid-urethra.

It is thus a feature of at least one embodiment of the invention to provide easier insertion of the pessary near the opening of the vagina rather than near the cervix and to provide support at the mid-urethra. Insertion of the present invention generally would not require a lubricant.

The pessary in a relaxed state may be bounded by a rectangle coplanar with the first and second axis and wider than it is tall. The pessary in a relaxed state may have a height and length with an aspect ratio of substantially 1:3.

It is thus a feature of at least one embodiment of the invention to use the shape of the pessary to translate the forces on the pessary to produce lateral forces to hold the pessary in place.

The pessary may provide a flexure such that the first and second ends may reversibly contact one another.

It is thus a feature of at least one embodiment of the invention to provide a pessary that does not require exact fit within the vagina by using an arch that springs out to conform to the vaginal canal.

The pessary may provide a flexure of one of the first and second ends of at least 3 cm with an oblique force of less than 200 grams when the other of the first and second ends is stationary.

It is thus a feature of at least one embodiment of the invention to apply minimal contact to the vaginal walls to increase comfort.

The pessary may be an elastomer that is a silicone rubber. The pessary may have a hardness between 30-40 durometer.

It is thus a feature of at least one embodiment of the invention to create a compact and adaptable pessary by using a material that can withstand repeated flexing with good resilience.

The pessary may have a length defined by the distance between the first and second ends of between 2.5-3.5 cm.

It is thus a feature of at least one embodiment of the invention to provide a smaller, more compact pessary. The pessary is sized to fit within and extend across the vagina of an average adult female human.

There may be additional pessaries providing a kit of pessaries, each pessary defining a different length for insertion in different sizes of vaginas.

It is thus a feature of at least one embodiment of the invention to provide multiple sizes for proper fitting in different sized vaginal canals, for example, for comfort, to prevent the pessary from falling out, and to apply the appropriate support force.

In another embodiment, the present invention may be a pessary device for treating stress urinary incontinence having a bridge support extending along a first axis between a first end and a second end of the bridge support and arching along a second axis perpendicular to the axis; a first grip portion at the first end and extending perpendicularly to the first and second axes and configured to engage a sidewall of a vagina; and a second grip portion at the second end and extending perpendicularly to the first and second axes and configured to latch onto an opposing sidewall of the vagina; where the bridge support is elastically deformable to bend at substantially a midpoint and to provide an outward force at the opposing first and second ends.

It is thus a feature of at least one embodiment of the invention to provide support knobs that provide a toe-hold grip on the vaginal walls and prevent torsion.

The first and second grip portions may have a cross section that is at least 1.5 times as wide as it is tall.

The pessary may have a height of the bridge support defined by the distance between a top of the mid-section and the bottom of the first and second ends of approximately 1 cm.

In another embodiment, the present invention may be a pessary device for treating stress urinary incontinence having a bridge dimensioned to extend across a human vaginal canal and having a first and second end configured to contact opposing sidewalls of the vaginal canal; where the bridge is made of a substantially incompressible elastomer and configured to flex at substantially a midpoint of the bridge to allow insertion of the device into the vagina and to unflex to a length greater than the width of the vaginal canal to allow the first and second end to grip opposing sidewalls of the vaginal canal.

It is thus a feature of at least one embodiment of the invention to use the natural resistance of the vaginal tissue to brace the pessary against the vaginal walls.

The bridge may form a substantially obtuse angle when inserted into the vagina.

The bridge may be configured to flex to form a substantially acute angle to allow insertion of the device into the vagina.

The midpoint of the bridge may contact the sidewall of the vaginal canal.

These particular objects and advantages may apply to only some embodiments falling within the claims and thus do not define the scope of the invention.

### BRIEF DESCRIPTION OF THE DRAWINGS

FIG. 1 is a perspective view of a pessary of the present invention shown generally from above;

FIG. 2 is a figure similar to FIG. 1 showing the pessary shown generally from below;

FIG. 3A-3D is a front elevation view, left side elevation view, bottom plan view, and right side elevation view of the pessary of FIG. 1;

FIG. 4A-4C is the pessary of FIG. 1 flexed at a mid-section before insertion of the device into the vagina, and showing three different sizes that would come in a pessary kit;

FIG. 5 is a cross-section of the female anatomy showing the pessary inserted near the opening of the vagina and showing the intra-abdominal pressures applied to the region;

FIG. 6 is an inferior view of the vaginal opening and showing the pessary inserted in the vagina and showing the forces applied to the urethra such that the pessary supports the mid-urethra;

FIG. 7 is a perspective view of the female anatomy showing the mid-urethra being supported from behind by the pessary inserted into the vagina;

FIG. 8 is an alternative embodiment of a pessary of the present invention taking a “U-shaped” configuration and a mesh spanning the two ends with suction cups at the pessary ends;

FIG. 9 is an alternative embodiment of a pessary of the present invention taking an inverted “U-shaped” configuration with bowed out arms and slightly concave mid-section; and

FIG. 10 is an alternative embodiment of a pessary taking a rounded “W-shaped” configuration.

### DETAILED DESCRIPTION OF THE INVENTION

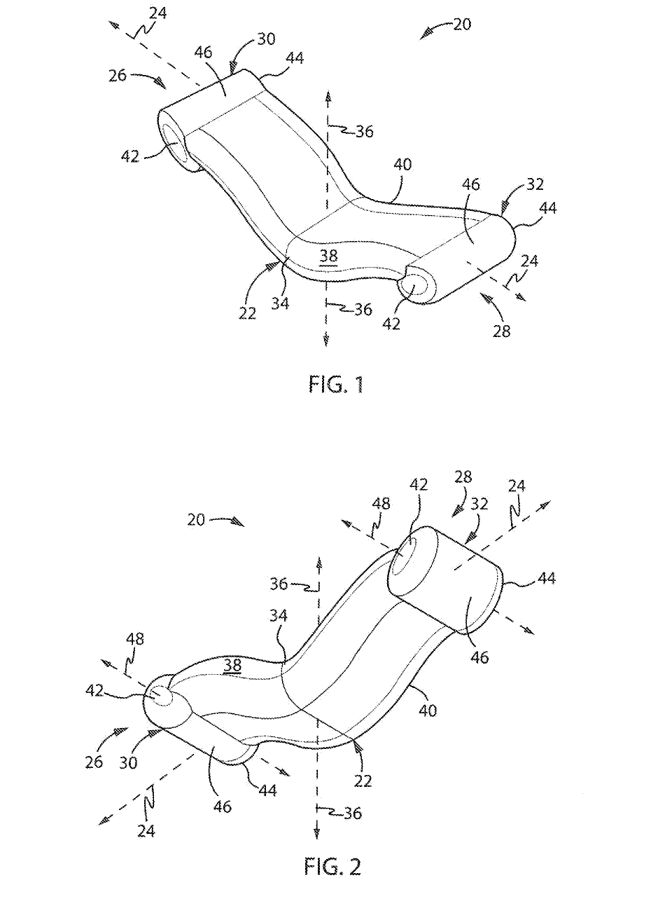

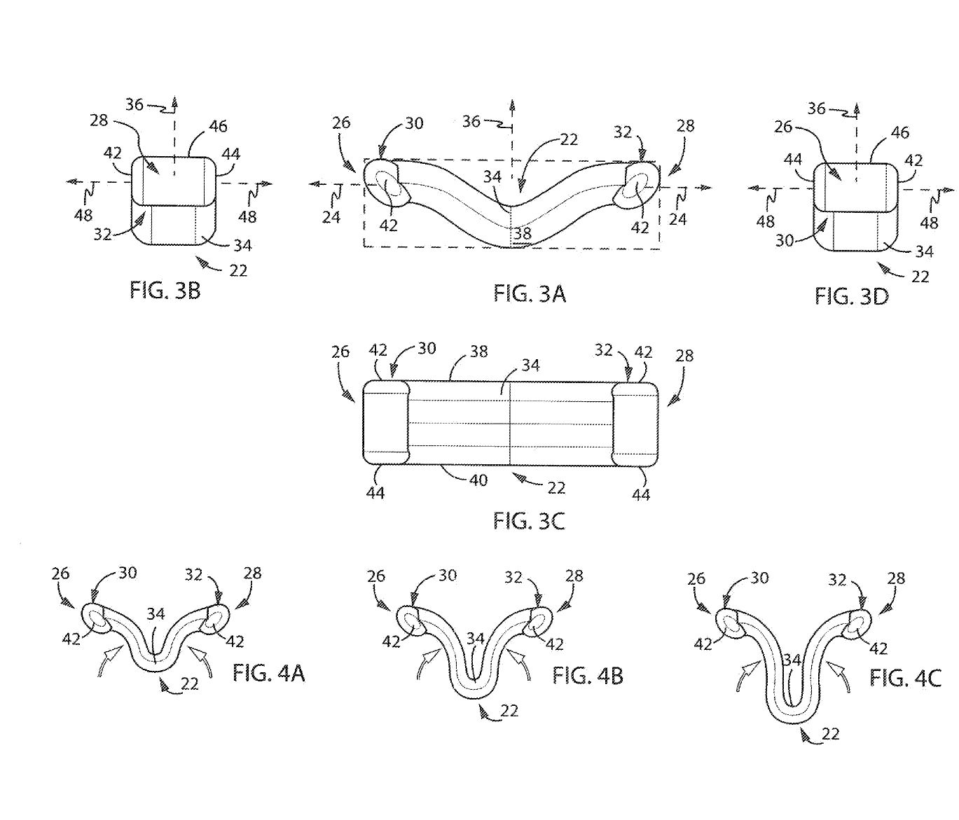

Referring now to FIGS. 1 and 2, a pessary 20, for example, of a substantially incompressible elastomer such as a medical-grade silicone, silicone rubber, or rubber, may be defined by a bridge 22 structure extending generally along an axis 24 and having a height of substantially 1.0 cm and between 1.0-2.0 cm, a length of substantially 3.0 cm and between 2.0-4.0 cm, and a width of substantially 1.0 cm and between 1.0-2.0 cm. A first end 26 of the pessary 20 may be positioned opposite a second end 28 of the pessary 20 with each end having support knobs 30, 32 for facilitating attachment to a vaginal wall 70, to be further described below. As used herein, incompressible means that during flexing, the volume of the pessary 20 is substantially constant as opposed to a foam material or the like. As such the pessary 20 when properly braced in an arched form can resist compression forces until a buckling point.

The pessary 20 may be a medical-grade silicone rubber commercially provided by, e.g., Dow Corning, Blue Star Silicone or Applied Silicone, which is biocompatible, easy to clean, and safe for medical applications. The medical-grade silicone rubber allows the material to flex at generally a mid-point when inward force is applied to the ends 26, 28 while rebounding back to its original form when the force is released. The medical-grade silicone may have a hardness of substantially 30-durometer and between 20-50 durometer. Optionally, an internal plastic component may be used inside the medical-grade silicone to increase the rigidity of the pessary 20. The resilience of the medical-grade silicone rubber allows for repeated flexing of the pessary 20 without losing its ability to rebound back to its original shape and size. It is contemplated that the pessary 20 may be durable enough to be used daily (for at least 12 hours) and to be taken out at night, for a duration of at least ninety days.

Referring also to FIGS. 3A-3D, in one embodiment of the invention the bridge 22 may take an inverted arch configuration, extending a distance between the first and second ends 26, 28. A mid-section 34 of the bridge 22 extends outwardly along an axis 36 substantially perpendicular to the axis 24 to form an inverted arched central portion. Opposite distal ends of the mid-section 34 are flanked by the first and second ends 26, 28 of the pessary 20, respectively. The first end 26 and second end 28 extend substantially along the axis 24 to provide generally opposing lateral forces to the perpendicularly extending mid-section 34.

The first end 26 may provide a first support knob 30 and the second end 28 may provide a second support knob 32 for facilitating an attachment to the vaginal walls 70. The support knobs 30, 32 may be defined as cylindrical pads with a body section 46 extending along an axis 48 of the cylinder perpendicular to axes 24, 36 and terminating at circular cross-sectional front 42 and rear 44 ends. The body section 46 extends along the width of the pessary 20 defined by a distance between a front side 38 and a rear side 40 of the pessary 20 with the front ends 42 substantially aligned with the front side 38 of the pessary and a rear ends 44 substantially aligned with the rear side 40 of the pessary. The support knobs 30, 32 may also extend past the front side 38 and rear side 40 to provide a longer gripping surface. The body section 46 of the support knobs 30, 32 provides a curved, outwardly protruded surface for comfortably applying concentrated pressure to the vaginal walls 70. When the body section 46 is cut along axis 48 perpendicular to axis 24, it has a cross section wider than it is tall, for example, 1.5-2 times wider than it is tall, to prevent torsion of the pessary 20 within the vaginal canal.

Referring to FIG. 3A, the pessary 20 may be sized such that in a relaxed state and when placed within a rectangle, the rectangle is wider than it is tall. The rectangle may have an aspect ratio of 3:1 or 3:2. The angle formed by the first and second ends 26, 28 may form an obtuse angle when un-flexed or relaxed, such as when installed within the vagina 52. When flexed, the angle formed by the first and second ends 26, 28 may form an acute angle, see FIG. 4A-4C.

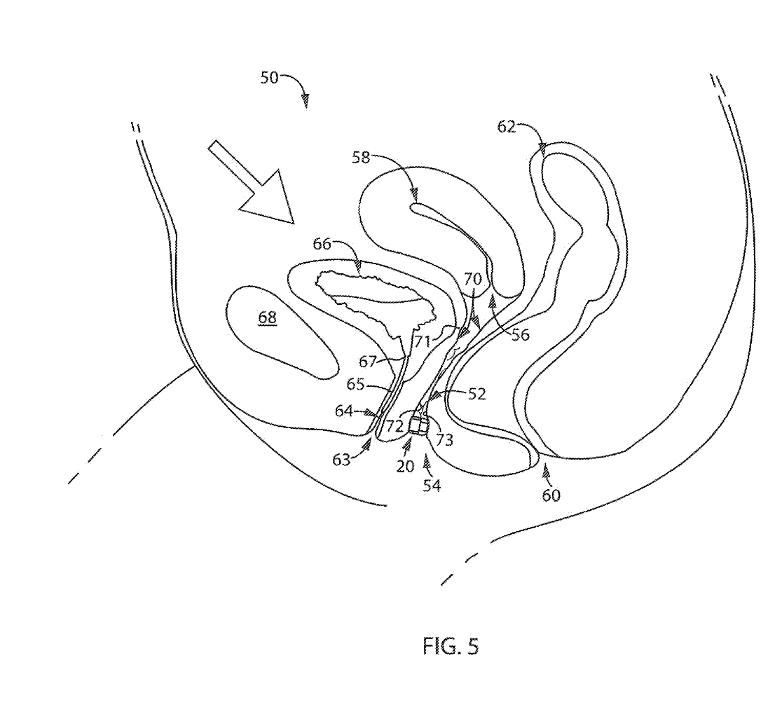

Referring to FIG. 5, a cross-sectional representation of the average female human anatomy 50 is shown with the “anterior” referring to the front of the human body, the “posterior” referring to the back of the human body, the “left lateral” referring to the left side of the human body, and the “right lateral” referring to the right side of the human body. As is generally understood in the art, the vagina 52 provides a passageway from an opening 54 or introitus of the vagina 52 to the cervix 56 of the uterus 58. The vagina 52 is defined by inner walls 70 providing an upper wall 72, lower wall 73, and left 74 and right 76 sidewalls. An anus 60 and rectum 62 are posterior to the vagina 52. A urethra 64 and urinary bladder 66 are anterior to the vagina 52. The urethra 64 provides a mid-urethral section 65 or sub-urethral section substantially mid-way between the urethral opening 63 and the urinary bladder 66, and a bladder neck 67 or base below the urinary bladder 66 but above the mid-urethral section 65. The pubic bone 68 is anterior to the urethra 64 and urinary bladder 66 and creates a notch 71 in the upper vaginal wall 72 behind the pubic bone 68 where it protrudes.

Referring to FIG. 4A-4C, the pessary 20 may be inserted within the human female vagina 52 by pinching or applying inward forces to the first and second ends 26, 28 so that the pessary 20 flexes at substantially a mid-point of the pessary bridge 22 and the first and second ends 26, 28 come together. The length of the pessary 20 when flexed is decreased such that the pessary 20 may be easily inserted into the vaginal opening 54.

Referring to FIG. 5, the pessary 20 may be inserted substantially 1 cm and between 1-2 cm into the opening 54 of the vagina 52. In another embodiment, the pessary 20 may be inserted substantially 2 cm and between 1-2 cm below the protrusion 71 created by the pubic bone 68. The pessary 20 is oriented such that the convex side of the mid-section 34 extends toward the lower wall 73 of the vagina 52 adjacent the rectum 62. The underside or concave side of the mid-section 34 faces the opposite upper wall 72 of the vagina 52 posterior and adjacent to the urethra 64. The first and second ends 26, 28 contact the left 74 and right 76 sidewalls of the vagina at the left and right laterals of the human body, respectively. Due to the symmetry of the pessary 20, it is understood that the pessary 20 may be rotated so that the first and second ends 26, 28 are switched and contact the opposite of the left and right sidewalls 74, 76 without significant difference. It is contemplated that the pessary 20 may be inverted so that the concave side of the mid-section 34 extends toward the lower wall 73 and the convex side faces the opposite upper wall 72. The pessary 20 may be inverted, for example, to apply greater passive support at the mid-urethral section 65.

Once the pessary 20 is inserted to the desired depth and orientation within the vagina 52, the force applied to the first and second ends 26, 28 are released so that the pessary 20 relaxes to its relaxed state and the support knobs 30, 32 contact the inner walls 70 of the vagina 52. The support knobs 30, 32 produce a “toe-hold” attachment to the inner walls 70 with minimal discomfort. The body section 46 may extend along the vaginal canal to prevent twisting of the pessary 20 or unwanted movement. The body section 46 also distributes the pressure placed on the vaginal walls 70.

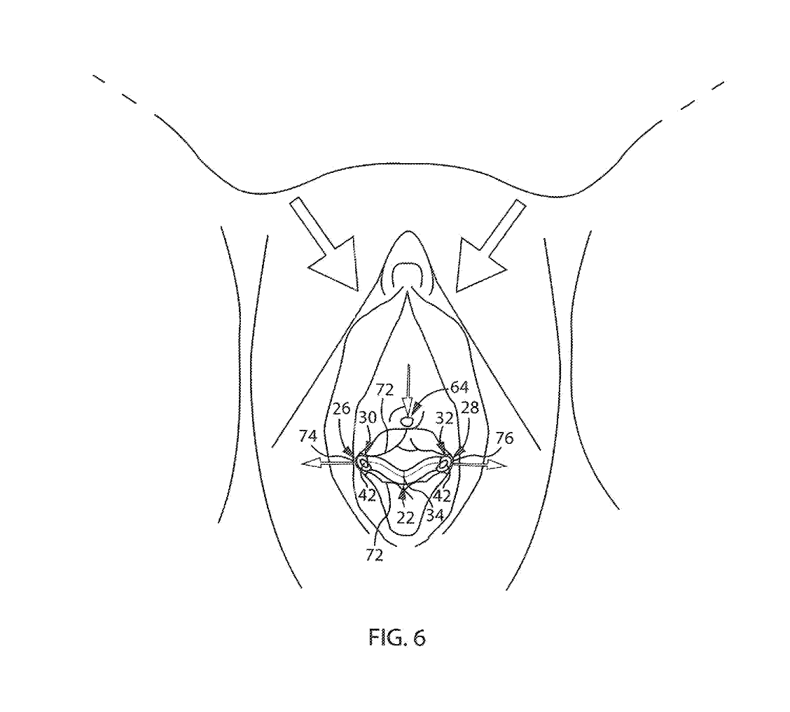

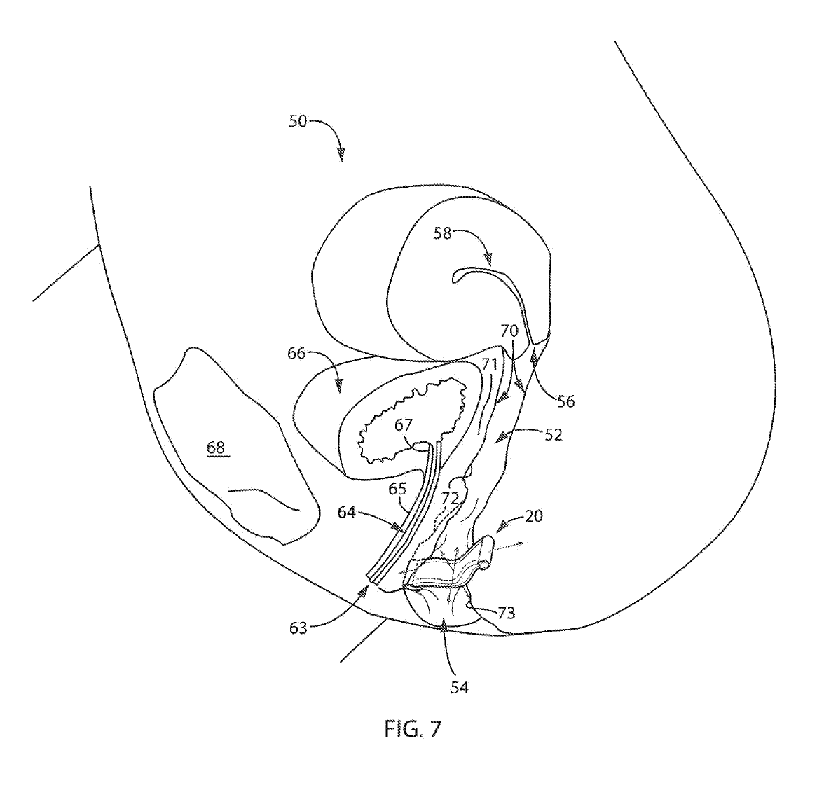

Referring also to FIGS. 6-7, when inserted in the vagina 52, the mid-section 34 of the pessary 20 is positioned substantially behind and below the mid-urethral section 65 of the urethra 64. In this position, the pessary 20 acts as a backstop to passively support the mid-urethra 65 underneath the mid-urethra 65 when intra-abdominal pressure places downward forces on the urethra, such as during physical activity. In this respect, the pessary bridge 22 acts as a substitute “sling” by supporting underneath the mid-urethra 65 through the upper vaginal wall 72 adjacent the urethra 64 instead of requiring a surgical sling extending around the mid-urethra 65 as found in mid-urethra sling surgery.

Referring to FIG. 6, the pessary 20 is able to brace itself against the vaginal walls 70 through the translation of forces against the mid-section 34 of the bridge 22 to the first and second ends 26, 28 and the slight elongation of the vagina 52. When the pessary 20 is inserted, the first and second ends 26, 28 extend outward laterally against the vaginal walls 70, utilizing the natural resistance of the vaginal walls 70 to produce a snug fit. When intra-abdominal pressure is applied to the urethra, additional forces are exerted at or near the mid-section 34 of the pessary 20 located behind the mid-urethra 65. The shape of the pessary 20 is able to translate the forces applied to the mid-section 34 to the generally opposing first and second ends 26, 28 in a generally perpendicular direction from the original forces. Thus, the forces applied to the mid-section 34 may be used to help maintain or support the pessary 20 at the walls 70 of the vagina 52. In this respect, the pessary 20 uses its own unique shape to prevent it from falling out or rotating from its original position without reliance on vaginal notches or a large or more expansive pessary 20 design.

By utilizing limited points of contact at the vaginal walls 70, the pessary 20 may provide increased comfort to the user. However, it is contemplated that additional support knobs may be added to the device to increase the points of contact along the vaginal walls 70. Also, the support knobs 30, 32 may be enlarged or extended in size to increase the contact area of the knobs against the vaginal wall 70, thus improving the hold of the pessary 20 to the vaginal wall 70.

The pessary 20 is sized to arch the vagina 52 near the opening 54 of the vagina 52 which may generally have a smaller diameter than deeper in the vagina 52. For example, an average width of the vaginal opening 54 in an average adult woman is approximately 2.62 cm, which is less than the average width at the pelvic diaphragm—2.78 cm—or at the proximal vagina-3.25 cm. Therefore, the pessary 20 may be smaller and less intrusive than pessaries that are configured to fit deeper within the vagina 52 where the width is generally greater than the introitus diameter. In this respect, the pessary 20 is sized slightly larger than the vaginal width so that it may be maintained within falling out but not too large as to exert too much pressure on the vaginal walls 70.

Referring to FIGS. 4A-4C, the present invention may provide a kit of more than one pessary 20, each pessary being a different size to accommodate varying widths of the vaginal opening. The user may select a particular pessary 20 depending on the fit of the pessary 20 in the vagina 52 so that it does not fall out or apply too much pressure. The kit may come with vaginal pessaries 20 of varying lengths, such as substantially 2.5 cm (small), 3 cm (medium) and 3.5 cm (large), or a length of between 1-2 cm larger than the introital diameter of the user's vagina.

Insertion of the pessary 20 may be facilitated by an applicator (not shown), which automatically flexes the pessary 20 before and during insertion into the vagina 52. Once the pessary 20 is inserted, the applicator may remove force on the pessary 20 ends to allow the pessary 20 to un-flex. The applicator may include an insertion member and a plunger. The insertion member may include the pessary 20, which is expelled by the plunger when the insertion member is in the proper position.

Removal of the pessary 20 may be facilitated by a cord or string (not shown) attached to the pessary 20 which allows the user to pull the string to remove the pessary 20 from the vagina 52. The string may be attached, for example, at the first and second ends 26, 28, respectively, such that pulling the string also causes the pessary 20 to flex facilitating the removal of the device.

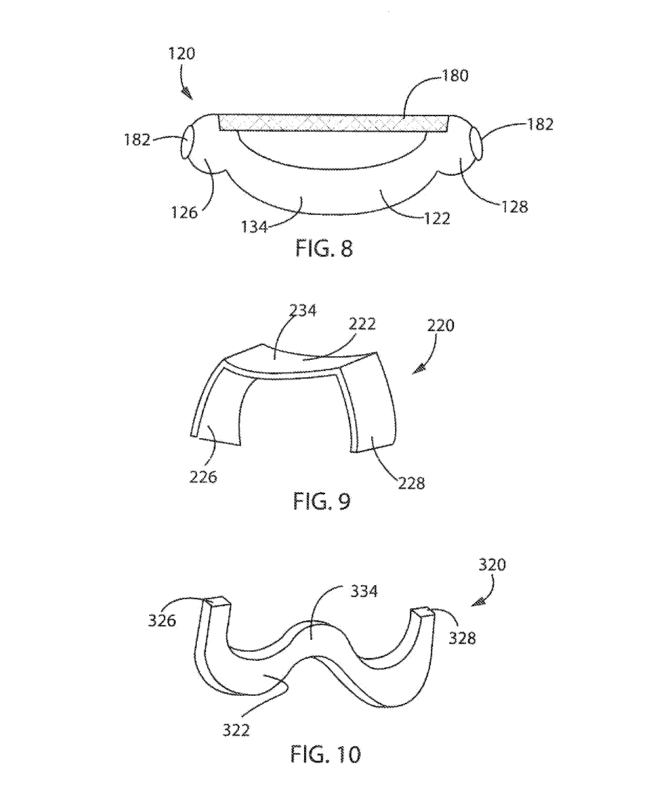

Referring to FIG. 8, in an alternative embodiment, the pessary 120 may take a bridge 122 configuration with a first 126 and second 128 end and a mid-section 134 which makes an inverted arch or “U-shape.” The ends 126, 128 may be flanked by a mesh 180 extending between the first 126 and second 128 ends to provide support on forces perpendicular to the mesh 180. The mesh 180 may be a flexible and thin medical-grade silicone.

Similarly, the pessary 120 may be folded at substantially a mid-section 134 of the bridge 122 by applying force to the ends 126, 128. The pessary 120 may be inserted such that the arched mid-section 134 extends toward the lower wall 73 of the vagina 52 and the mesh 180 extends toward the upper wall 72 of the vagina 52. The first and second ends 26, 28 contact the left 74 and right 76 sidewalls of the vagina at the left and right laterals of the human body, respectively. The mesh 180 is positioned behind and under a mid-urethral section 65 of the urethra 64 so as to provide passive support to the mid-urethra 65 when under pressure. It is understood that mesh 180 may used in any of the pessary embodiments described herein.

In the alternative embodiment, the ends 126, 128 may also include suction cups 182 to assist in the attachment of the ends 126, 128 to the vaginal wall 70. It is understood that suction cups 182 may be applied to the ends of any of the pessary embodiments described herein.

Referring to FIG. 9, in an alternative embodiment, the pessary 220 may take a bridge 222 configuration with a generally straight or slightly concave mid-section 234. The mid-section 234 may be flanked by downwardly extending first 226 and second 228 ends. The first 226 and second 228 ends may be bowed outward to provide a generally concave shape in accordance with an outline of the vaginal walls 70. The first and second 226, 228 ends way extend a distance at least half the length of the pessary 220 to provide a greater attachment surface than the other embodiments.

Again, the pessary 220 may be folded at substantially a mid-section 234 of the bridge 222 by applying force to the ends 226, 228. The pessary 220 may be inserted such that the straight or concave mid-section 234 extends toward the upper wall 72 of the vagina 52 and the first and second ends 226, 228 contact the left 74 and right 76 sidewalls of the vagina at the left and right laterals of the human body, respectively. The mid-section 234 of the pessary 220 is positioned behind and under a mid-urethral section 65 of the urethra 64 so as to provide passive support to the mid-urethra 65 when under pressure.

Referring to FIG. 10, in an alternative embodiment, the pessary 320 may take a bridge 322 configuration with a generally curved “W-shaped” configuration whereby the arched middle section of the “W” extends upwardly to provide a curved mid-section 334.

Again, the pessary 320 may be folded at substantially a curved mid-section 334 of the bridge 322 by applying force to the ends 326, 328. The pessary 320 may be inserted such that the curved mid-section 334 extends toward the upper wall 72 of the vagina 52 and the first and second ends 326, 328 extend toward the upper wall 72 of the vagina to generally contact the left 74 and right 76 sidewalls of the vagina at the left and right laterals of the human body, respectively. The mid-section 234 of the pessary 220 is positioned behind and under a mid-urethral section 65 of the urethra 64 so as to provide passive support to the mid-urethra 65 when under pressure. The first and second ends 326, 328 may be inserted such that the ends 326, 328 latch into indentations of the upper wall 72 of the vagina 52.

Certain terminology is used herein for purposes of reference only, and thus is not intended to be limiting. For example, terms such as “upper”, “lower”, “above”, and “below” refer to directions in the drawings to which reference is made. Terms such as “front”, “back”, “rear”, “bottom” and “side”, describe the orientation of portions of the component within a consistent but arbitrary frame of reference which is made clear by reference to the text and the associated drawings describing the component under discussion. Such terminology may include the words specifically mentioned above, derivatives thereof, and words of similar import. Similarly, the terms “first”, “second” and other such numerical terms referring to structures do not imply a sequence or order unless clearly indicated by the context.

When introducing elements or features of the present disclosure and the exemplary embodiments, the articles “a”, “an”, “the” and “said” are intended to mean that there are one or more of such elements or features. The terms “comprising”, “including” and “having” are intended to be inclusive and mean that there may be additional elements or features other than those specifically noted. It is further to be understood that the method steps, processes, and operations described herein are not to be construed as necessarily requiring their performance in the particular order discussed or illustrated, unless specifically identified as an order of performance. It is also to be understood that additional or alternative steps may be employed.

It is specifically intended that the present invention not be limited to the embodiments and illustrations contained herein and the claims should be understood to include modified forms of those embodiments including portions of the embodiments and combinations of elements of different embodiments as come within the scope of the following claims. All of the publications described herein, including patents and non-patent publications, are hereby incorporated herein by reference in their entireties.

What we claim is:

1. A method of treating stress urinary incontinence comprising the steps of:
providing a pessary dimensioned to extend across opposing walls of a vagina and having a first end having a first support knob opposite a second end having a second support knob and a bridge extending along a first axis and arching along a second axis substantially perpendicular to the first axis between the first and second ends to provide a substantially incompressible structure configured to bend allowing pinching of the first and second ends together;
pinching the first and second ends together to allow insertion into the vagina;
inserting the pessary approximately 1-2 cm within the opening of the vagina; and
releasing the first and second ends of the pessary while the pessary is in the vagina to allow the first and second support knobs to separate to contact opposing walls of the vagina.

2. The method of claim 1 wherein the pessary in a relaxed state is bounded by a rectangle coplanar with the first and second axis and wider than it is tall.

3. The method of claim 1 wherein the height and length have an aspect ratio of substantially 1:3.

4. The method of claim 1 wherein the pessary provides a flexure such that the first and second ends may reversibly contact one another.

5. The method of claim 1 wherein the pessary provides a flexure of one of the first and second ends of at least 3 cm with an oblique force of less than 200 grams when the other of the first and second ends is stationary.

6. The method of claim 1 wherein the pessary is a silicone rubber.

7. The method of claim 1 wherein the hardness of the substantially incompressible structure is between 30-40 durometer.

8. The method of claim 1 wherein the bridge arches toward a posterior sidewall of the vagina to allow an underside of the bridge to support the mid-urethra.

9. The method of claim 1 further providing additional pessaries providing a kit of pessaries, each pessary defining a different length for insertion in different sizes of vaginas.

10. A pessary device for treating stress urinary incontinence comprising:
a bridge support extending along a first axis between a first end and a second end of the bridge support and arching along a second axis perpendicular to the axis;
a first grip portion at the first end and extending perpendicularly to the first and second axes and configured to engage a sidewall of a vagina;
a second grip portion at the second end and extending perpendicularly to the first and second axes and configured to latch onto an opposing sidewall of the vagina; and
wherein the bridge support is elastically deformable to bend at substantially a midpoint and to provide an outward force at the opposing first and second ends.

11. The device of claim 10 wherein the first and second grip portions have a cross section that is at least 1.5 times as wide as it is tall.

12. The device of claim 11 wherein the hardness of the bridge is between 30-40 durometer.

13. The device of claim 10 wherein a length of the bridge support defined by the distance between the first and second end is between 2.5-3.5 cm.

14. The device of claim 10 wherein the height of the bridge support defined by the distance between a top of the mid-section and the bottom of the first and second ends is approximately 1 cm.

15. A pessary device for treating stress urinary incontinence comprising:
a bridge dimensioned to extend across a human vaginal canal and having a first and second end configured to contact opposing sidewalls of the vaginal canal;
wherein the bridge is made of a substantially incompressible elastomer and configured to flex at substantially a midpoint of the bridge to allow insertion of the device into the vagina and to unflex to a length greater than the width of the vaginal canal to allow the first and second end to grip opposing sidewalls of the vaginal canal.

16. The device of claim 15 wherein the bridge forms a substantially obtuse angle when inserted into the vagina.

17. The device of claim 15 wherein the bridge is configured to flex to form a substantially acute angle to allow insertion of the device into the vagina.

18. The device of claim 15 wherein the midpoint of the bridge contacts the sidewall of the vaginal canal.

19. The device of claim 15 wherein the pessary is bounded by a rectangle wider than it is tall.

20. The device of claim 15 wherein the height and length have an aspect ratio of substantially 1:3.
